(19) United States
(12) Patent Application Publication
(10) Pub. No.: US 2005/0106270 A1
(43) Pub. Date: May 19, 2005

(54) Chemical treatment of in vivo tissue to alter charge and net charge density characteristics

(76) Inventors: Dale Paul Devore, Chelmsford, MA (US); Braden Patrick Devore, Las Vegas, NV (US)

Correspondence Address: BARLOW, JOSEPHS & HOLMES, LTD., 101 DYER STREET, 5TH FLOOR, PROVIDENCE, RI, 02903, US

(21) Appl. No.: 10/959,740

(22) Filed: Oct. 6, 2004

Related U.S. Application Data
(60) Provisional application No. 60/509,014, filed on Oct. 6, 2003.

(51) Int. Cl. A61K 33/00

(52) U.S. Cl. 424/722

(57) ABSTRACT

A method for treating animal tissue with acylation agents to alter the net charge and net charge density of the treated tissue for therapeutic applications is provided. The method involves applying an alkaline solution to the exposed tissue surface area. This results in deprotonation of ε-amino groups of lysine residues on the exposed tissue proteins so that the tissue proteins have a net charge. Then, an acylating agent is applied and the acylating agent reacts with the tissue protein to form a protein complex having an altered net charge. Acylating agents such as sulfonic acids, sulfonyl chlorides, and acid chlorides can be used. The method can be used to treat a wide variety of human tissues including the human cornea for correcting myopia. The method can also be used to treat skin tissue, so that there is an increase in dermal thickness and pliability. The method can be further used to treat articular cartilage.

### CROSS-REFERENCE TO RELATED APPLICATIONS

This application claims the benefit of U.S. Provisional Application No. 60/509,014 having a filing date of Oct. 6, 2003, the entire contents of which are hereby incorporated by reference.

### FIELD OF THE INVENTION

The present invention relates to a process for selectively treating in vivo animal tissue to alter its net charge and net charge density properties. More particularly, this invention relates to a process for selectively treating in vivo animal tissue by acylation to alter its net charge properties. This process may be used to increase the net negative charge on reacted tissue proteins, thereby increasing water binding characteristics of selectively treated in vivo animal tissue. Selective treatment of the peripheral circumference of the human cornea will result in selective swelling of the peripheral circumference causing flattening of the central corneal area, thereby providing a method to treat myopia. Such treatments of skin will result in an increase in dermal thickness and pliability. Such treatments may result in an improvement of articular cartilage quality. This process may also be used to decrease the net negative charge and net charge density in treated tissues.

### BACKGROUND OF THE INVENTION

It is known that various chemical agents will react with proteins to alter their chemical and physical characteristics. Generally, these chemical agents are used to modify proteins in solution. Several reviews discussing chemical modification are available including Chemical Reagents for Protein Modification, Ed. R L Lunblad, CRC Press, Boca Raton, 1991 and G R Stark, Recent Developments In Chemical Modification And Sequential Degradation Of Proteins, Advances in Protein Chemistry, 24: 261-308, 1970. Specific chemical agents react with deprotonated free amines on proteins to replace the positive (NH₃⁺) charge with a chemical moiety exhibiting a negative charge or neutral charge. Other chemical agents react with deprotonated amines on proteins to replace a single positive (NH₃⁺) charge with two positive charges (NH₃⁺×2). This change in net charge and charge density alters both the chemical and physical characteristics of the protein.

Acylation reactions have commonly been used to derivatize soluble and insoluble collagen and have been described by DeVore, et.al. in a series of patents (U.S. Pat. Nos. 4,713,446; 4,851,513; 4,969,912; 5,067,961; 5,104,957; 5,201,764; 5,219,895; 5,332,809; 5,354,336; 5,476,515; 5,480,427; 5,631,243; and 6,161,544). However, none of these patents describe the use of acylation agents to selectively alter the net charge and charge density of intact tissues for therapeutic applications. An increase in net negative charge density will increase water binding resulting in tissue swelling. A decrease in net negative charge will decrease water binding. Changes in net charge density also have dramatic effects on mechanical properties of treated tissues.

In the present invention, acylation reactions have been used to treat intact tissue to change the net charge on tissue proteins resulting in a dramatic change in chemical and physical properties. Sulfonic acids, anhydrides, sulfonyl chlorides, and acid chlorides are classes of chemical compounds that react with free amines of proteins resulting in the covalent attachment of the specific chemical moieties to proteins. These compounds are commonly known as acylation reagents.

Specific acylation agents have been used to alter the net charge and charge density of intact tissue proteins. Certain agents can be used to change the net charge from positive to negative. These agents include, but are not limited to, anhydrides including maleic anhydride, succinic anhydride, glutaric anhydride, citractonic anhydride, methyl succinic anhydride, itaconic anhydride, methyl glutaric anhydride, dimethyl glutaric anhydride, phthalic anhydride, and many other such anhydrides. Acid chlorides include, but are not limited to, oxalyl chloride, malonyl chloride, and many others. Sulfonyl chlorides include, but are not limited to, chlorosulfonylacetyl chloride, chlorosulfonylbenzoic acid, 4-chloro-3-(chlorosulfonyl)-5-nitroebnzoic acid, 3-(chlorosulfonyl)-P-anisic acid, and others. Sulfonic acid include, but are not limited to, 3-sulfoebnzoic acid and others.

Certain agents can change the net charge from one positive to two negatives per reacted site. Specific agents include, but are not limited to, 3,5-dicarboxybenzenesulfonyl chloride and others.

Certain agents can be used to change the net charge from positive to neutral per reacted site. Specific agents include, but are not limited to, anhydrides including acetic anhydride, chloroacetic anhydride, propionic anhydride, butyric anhydride, isobutyric anhydride, isovaleric anhydride, hexanoic anhydride, and other anhydrides; acid chlorides including acetyl chloride, propionyl chloride, dichloropropionyl chloride, butyryl chloride, isobutyryl chloride, valeryl chloride, and others; sulfonyl chlorides including, but not limited to, ethane sulfonyl chloride, methane sulfonyl chloride, 1-butane sulfonyl chloride, and others.

Certain agents can be used to change the net charge from one positive to two positives per reacted site. Specific agents include, but are not limited to, 4,6-diamino-2-methylthiopyrimidine-5-sulfonic acid, and others.

Alterations in the net charge or net charge density can affect hydration and mechanical properties of tissue. Specific changes in net charge and net charge density are proposed as therapeutic treatments for vision correction, skin rejuvenation, and improvements in the articular cartilage quality.

### SUMMARY OF THE INVENTION

The present invention is based on the discovery that chemical acylation of intact tissues can be conducted in a controlled manner to alter the net charge and net charge density of reacted tissues and tissue surfaces to provide specific therapeutic benefits or to rejuvenate tissues degenerated by aging and disease.

The inventors have demonstrated that acylation of intact tissue using specific agents can increase the net negative charge density resulting in an increase in tissue thickness and an increase in both low and high modulus measured from stress-stain analysis. Increased modulus readings relate to increased stiffness of treated tissues and more force required to compress the treated tissues. The inventors have also demonstrated that acylation of intact tissue using specific agents can decrease the net negative charge density resulting in negligible effect on tissue thickness but with dramatic reductions in low modulus data from stress-strain analysis. The latter relates to increased softening of treated tissue or less force required to compress the treated tissues.

The present invention features a process for reacting specific acylation agents with intact tissues or tissue surfaces to alter the net charge and net charge density of the treated tissue for therapeutic applications. The method includes steps of: (1) applying a treatment device to the tissue surface such that the desired area of the tissue surface is exposed to treatment solutions; (2) pretreating the exposed tissue surface with slightly alkaline buffer solution for 1-2 minutes to bring the pH of the tissue surface to between about 7.5 and about 9.5 resulting in deprotonation of e-amino groups of lysine residues on exposed proteins; (3) removing the pretreatment buffer solution using an absorbent sponge; (4) applying the chemical agent (acylating agent at a concentration of between 0.1 mg/mL and 100 mg/mL, preferably between 10 mg/mL and 50 mg/mL, in the same slightly alkaline buffer used in the pretreatment solution) to the exposed area such that the chemical agent immediately reacts with the exposed, pretreated tissue surface resulting in covalent bonding of the pendant chemical moiety to the deprotonated ε-amino groups of lysine residues on exposed proteins; (5) thorough rinsing of the total tissue surface to remove unreacted chemical agent and masking the deprotonated free amino group with the desired pendant group to alter the net charge and the net charge density of the treated tissue. The predominant protein to react with the acylation chemicals is collagen.

The primary tissues to be treated include skin, cornea, sclera tissue, conjunctival tissue, and articular cartilage. In the case of skin, acylation agents are applied to increase the net negative charge and the net charge density resulting in an increase in tissue hydration producing a thicker dermal skin layer with increased pliability. This treatment results in rejuvenation of thin, brittle skin. While many skin treatments are currently available in the form of cosmetic crèmes, none of these treatments specifically undergoes chemical reactions with dermal components causing an increase in chemical binding of water.

In the case of the cornea, acylation agents intended to increase net negative charge are applied to a selected circumferential ring on the periphery of the cornea to induce controlled peripheral stroma swelling and subsequent central cornea flattening. This non-surgical treatment can be used to correct myopia by flattening the cornea and reducing the diopters power of the central cornea. Surgical treatments to expand the periphery of the corneal stroma, thereby flattening the central cornea, have not been widely accepted (see U.S. Pat. Nos. 5,188,125; 5,300,118; 5,312,424; 5,318,047; 5,466,260; 5,888,243; 6,206,919; 6,228,114; and 6,511,508). Treatments with acylation agents reducing the net negative charge do not result in controlled peripheral swelling, but do cause demonstrable tissue softening. These agents have been reacted with deprotonated amine groups on sclera proteins causing demonstrable softening of sclera tissues. This treatment has therapeutic potential for reversal of presbyopia. In such cases, the sclera becomes more pliable and extensible. Several patents by Schachar describe methods of treating presbyopia using sclera expansion techniques (U.S. Pat. Nos. 5,354,331; 5,465,737; 5,489,299; 5,503,165; 5,529,076; 5,722,952; 6,007,578; 6,280,468; and 6,299,640).

In the case of cartilage, acylation agents can be used to alter the net charge density by treating intact articular cartilage with agents that increase the net negative charge density or with agents that reduce the net negative charge density (subsequent increase in the ratio of positive charge density to negative charge density). Specific treatments may have therapeutic applications in rejuvenating aged and diseased articular cartilage.

### BRIEF DESCRIPTION OF THE FIGURES

The novel features that are characteristic of the present invention are set forth in the appended claims. However, the preferred embodiments of the invention, together with further objects and attendant advantages, are best understood by reference to the following detailed description taken in connection with the accompanying Figures in which:

FIG. 1 is a topographical photograph of an enucleated porcine eye specimen prior to treating the specimen with an acylating agent in accordance with the method of this invention;

FIG. 2 is a topographical photograph of an enucleated porcine eye specimen after treating the specimen with an acylating agent in accordance with the method of this invention;

FIG. 3 is a topographical photograph of an in vivo cat eye specimen prior to treating the specimen with an acylating agent in accordance with the method of this invention;

FIG. 4 is a topographical photograph of an in vivo cat eye specimen after treating the specimen with an acylating agent on Day 1 and Day 7 in accordance with the method of this invention; and

FIG. 5 is a graph showing the stress-strain analysis of dermal tissue specimen C and dermal tissue specimen D after treating the specimens with the same acylating agent under different treatment conditions.

### DETAILED DESCRIPTION OF THE INVENTION

By “acylating agent,” it is meant an agent that transfers an acyl group to another nucleophile. Examples of acylation agents are sulfonic acids, anhydrides, sulfonyl chlorides, and acid chlorides.

A listing of appropriate anhydrides, acid chlorides, sulfonyl chlorides, and sulfonic acids can be found in the Sigma-Aldrich Chemical company catalogue.

“Superficial surface” is meant to be the very top layer of tissues, to depths of about 2 to 50 microns.

The present invention provides methods for selectively treating intact tissue in a controlled manner to alter the net charge and net charge density of reacted tissues and tissue surfaces to provide specific therapeutic benefits or to rejuvenate tissues degenerated by aging and disease.

The present invention describes methods for applying specific acylating agents to intact tissues and tissue surfaces, following pretreatment with a slightly alkaline solution, to react with deprotonated free amines on tissue proteins, thereby altering the net charge characteristics and charge density of the reacted tissue.

In general, the acylating agent may include agents in the form of sulfonic acids, anhydrides, sulfonyl chlorides, and acid chlorides. Concentrations of the acylation agents range from 0.1 mg/mL to 100 mg/mL, preferably from 1 mg/mL to 50 mg/mL.

Prior to the addition of the acylating reagent, the tissue is pretreated with a solution exhibiting a pH from about 7.5 to about 9.5. The solution may be composed of a single component, such as disodium phosphate, sodium pyrophosphate, or sodium borate, or may be a buffer composition providing a pH ranging from about 7.5 to about 9.5. The concentration of the alkaline solution ranges from 0.01M to 0.2M.

In the case of corneal treatment to treat myopia, the device for applying the acylating agent to the corneal surface is composed of a series of concentric circles. The center of the concentric circles is solid and is seated on the corneal apex, preventing exposure of the central cornea to the sulfonic acid dye or stain. An intermediate concentric circle is open to the surface of the cornea allowing exposure of this surface only to the acylating agent. The outer circle is also solid and seated firmly on the corneal surface preventing exposure of the corneal surface to the acylating agent. The width of the intermediate concentric circle can be adjusted to allow exposure of the corneal surface to predetermined widths of the acylating agent. Thus, a ring of predetermined width can be formed on the corneal surface for specific therapeutic applications. The exposed corneal tissue is generally a peripheral ring, approximately 2 mm in diameter near the limbus of the corneal surface. In one design, a port is fabricated fitting the end of a 1.0-2.5 cc syringe. Acylation solution is injected into the open ring through this delivery port to treat the exposed tissue surface. The acylating agent is also removed using this port and rinse solutions applied to remove unbound dye or stain. The extent of tissue treatment is dependent on the concentration of the acylating agent, exposure time, and the pH of the exposed tissue. Other configurations for delivery devices can be fabricated to treat predetermined regions of the cornea surface. For example, a dry sponge, pre-dosed with an appropriate amount of acylating agent is fabricated to specific dimensions such that when the dry, pre-dosed sponge is wet, the chemical agent is delivered to the desired exposed tissue surface. The dry, pre-dosed sponge is fabricated in the form of a thin ring. The ring is then placed in a delivery device. Fluid is then applied to the ring causing it to wet and instantly deliver the pre-dosed acylating agent to the exposed tissue surface to form an exposure ring in the same dimensions as the delivery ring. The delivery ring may be fabricated in different dimensions, thickness and diameter to treat the corneal tissue.

As discussed above, the method of this invention is used to treat animal tissue, particularly human tissue in vivo and comprises the steps of: (a) providing an exposed surface area of animal tissue; (b) applying an alkaline solution, preferably a solution having a pH in the range of about 7.5 to about 9.5, to the exposed tissue surface area so that tissue proteins having a net charge are formed (the treatment with the alkaline solution results in deprotonation of ε-amino groups of lysine residues on the exposed tissue proteins); and (c) applying an acylating agent such as, for example, a sulfonic acid, sulfonyl chloride, or acid chloride to the exposed tissue surface areas so that the acylating agent reacts with the tissue proteins to form protein complexes having a different net charge than the net charge of the tissue proteins formed in above-described step (b).

The features and other details of the invention will now be more particularly described and pointed out in the following examples describing preferred techniques and experimental results. These examples are provided for the purpose of illustrating the invention and should not be construed as limiting the scope of the invention.

### EXAMPLES

Corneal Reshaping—Treatment of Myopia

### Example 1

### Enucleated Porcine Eyes

Eyes were procured from a local slaughterhouse, positioned in a device to stabilize the eye and subjected to topographical evaluation using the Optikon 2000 system. The corneal surface was dried using sterile gauze and then wetted with drops of buffer solution. The wetted eyes were again dried and exposed again to the same solution. Then a peripheral ring around the circumference of the corneal surface, slightly away from the limbus and the central cornea, was carefully treated by adding drops of buffer containing the active agent, a 20 mg/ml solution of glutaric anhydride. The eyes were then reexamined topographically and photos taken. Following evaluation, the eyes were placed in OptiSol for storage pending additional evaluations. Three eyes were treated using this protocol. In two eyes the active agent at 20 mg/mL was applied to a ring around the corneal periphery. The exposure width was approximately 1 mm. In one eye the active agent at 20 mg/mL was applied to a 2 mm diameter area on the apex of the central cornea. The exposure time was 1 minute. All eyes were then washed with neutral pH phosphate buffer.

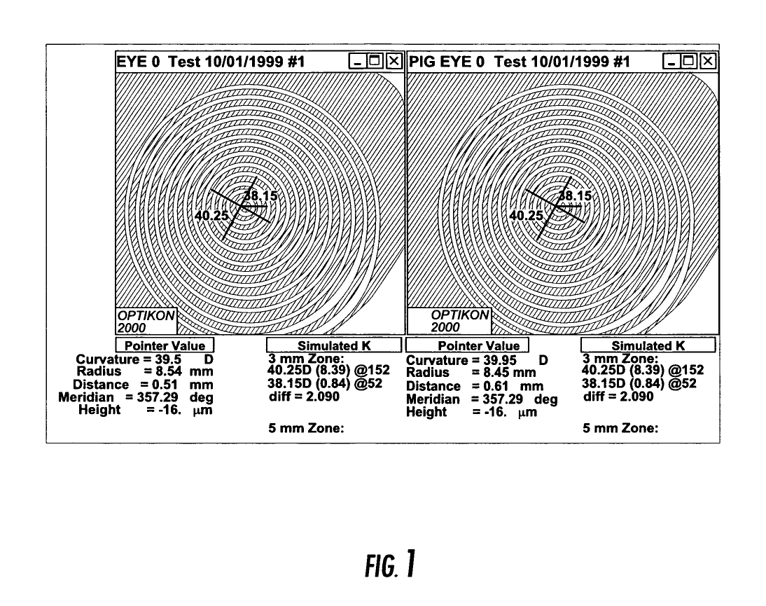

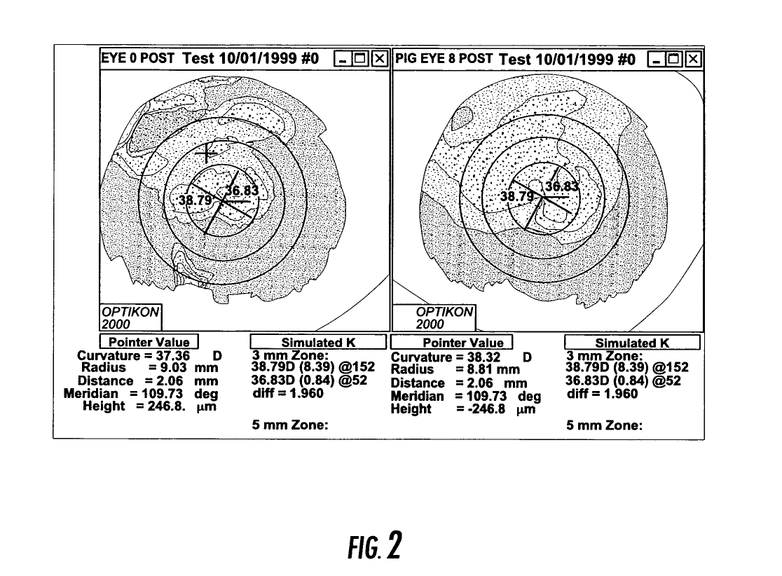

As reported in the following Table 1, topographical evaluation showed that treatment of the periphery of the cornea with the active agent reduced corneal power of porcine eyes by more than 2 diopters. Treatment of the central cornea increased the refractive power by about 0.7 diopters. All eyes appeared clear by visual examination. Pre and post-treatment topographical photographs of enucleated eye #2 are shown in FIGS. 1 and 2.

TABLE 1
Corneal Power (Diopters) As Measured By Topographical Mapping

| Animal Number | Pre-treatment Power (D) | Post-treatment Power (D) |
|---|---|---|
| #1-central cornea | 40.6 | 41.3 |
| #2-peripheral | 39.5 | 37.4 |
| cornea | | |
| #3-peripheral | 43.6 | 40.9 |
| cornea | | |

These results demonstrate that careful treatment of the peripheral corneal surface can result in significant central corneal flattening. Treatment of the central cornea resulted in minor steepening. It is believed that these effects result from controlled hydration of the treated surface.

This simple technique may revolutionize methods used to treat refractive errors. Treatment of areas in the central cornea appear to result in corneal steepening thus providing a simple method to treat hyperopia. Treatment of selective areas of the corneal surface may furthermore be effective in treating astigmatism.

### Example 2

### In Vivo Cat Model

Two cats were treated with the active agent, glutaric anhydride. Treatment was applied to the right eye (OD) while the contra lateral eye (OS) served as a control. Buffer solution (0.02M disodium phosphate solution at pH 9.0) was first applied to the corneal surface. This was immediately followed by application of a solution of glutaric anhydride in disodium phosphate into a peripheral ring of a corneal mold placed on the corneal surface. The mold provided a tight seal to prevent migration of the active agent to the central cornea. Two treatment applications were provided at Day 1 and Day 7. The dosage of the active agent was 50 mg/mL. Eyes were examined for another 7 days following the second treatment.

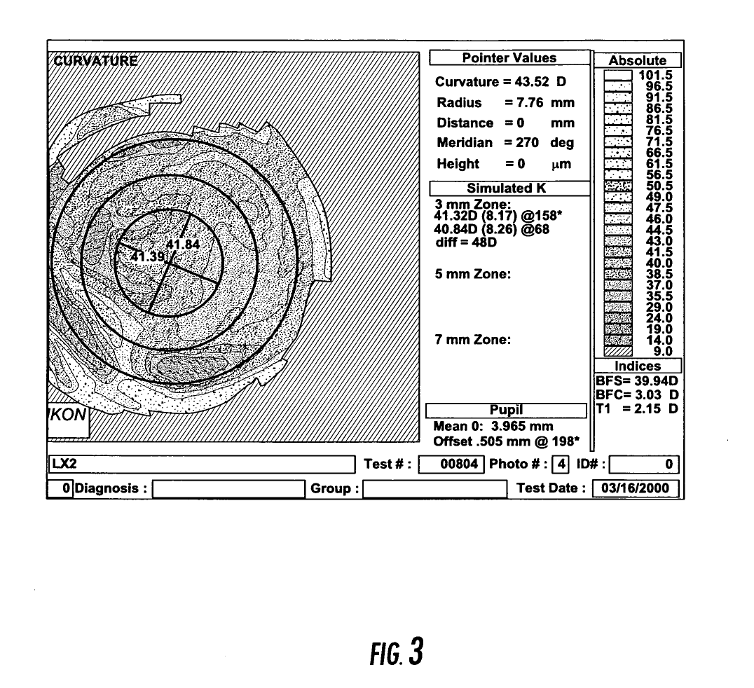

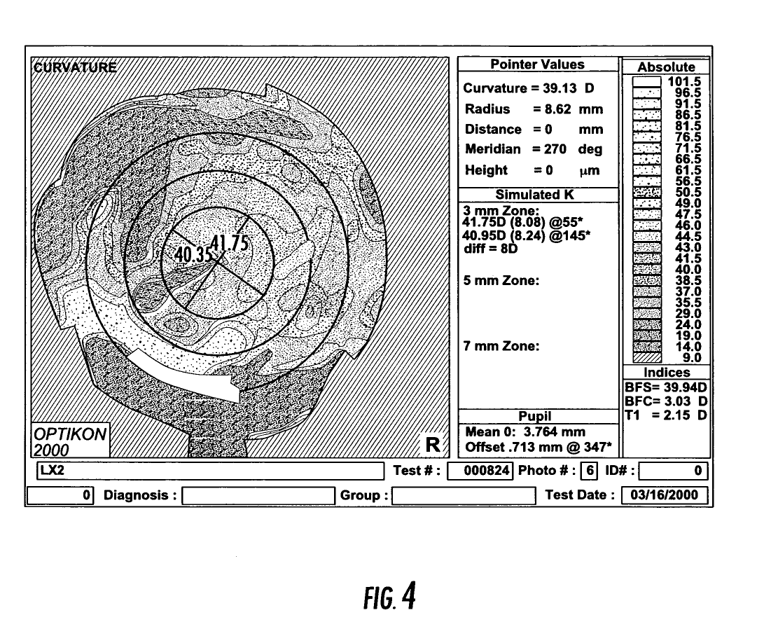

As reported in the following Table 2, results from topographical evaluation show that refractive power (D) of the treated eye for Cat 1 reduced from 43.65 to 38.88 (4.77 Diopters). Results for Cat 2 showed a reduction from 42.83 to 40.68 (2.2 Diopters). Optical examinations, including slit-lamp biomicroscopy and Shiotz tonometry, showed no differences between treated and control eyes 7 days following the second treatment. FIGS. 3 and 4 show the topographical maps for the treated eye (OD) of Cat 1.

TABLE 2
Corneal Power (Diopters) As Measured By Topographical Mapping

| Animal Number | Average Pre-treat (D) | Average Post-treat (D) |
|---|---|---|
| Cat 1 | 43.65 | 38.88 |
| Cat 2 | 42.83 | 40.68 |

These results appear to confirm preliminary studies showing that the application of a specific active agent can reduce the curvature of the central cornea.

### Example 3

### Treatment of Enucleated Porcine Eyes

Whole, fresh porcine (pig) eyes were obtained from a local abattoir and immediately placed in Optisol GS preservation solution. The whole eye was placed in a holder allowing the corneal surface to be exposed. A 7 mm trephine was used to cut through the epithelium and penetrate the superficial corneal tissue. The corneal surface was then flooded with 0.2M disodium phosphate solution, pH 9.0. After 1 minute, the surface of the cornea was dried using an absorbent wipe (Kim Wipe). The corneal surface was immediately treated with 0.2M disodium phosphate solution, pH 9.0, containing 50 mg/mL of glutaric anhydride. After 1 minute of exposure, the cornea was flushed with phosphate buffered saline, pH 7.2. The surface of the cornea was inspected and the corneal curvature examined and compared to untreated eyes. A white ring was observed at the trephine impression, even after several days. The central corneal surface was clearly depressed or flattened compared to untreated eyes. The application of glutaric anhydride to a trephined peripheral ring of a pig cornea produced obvious flattening of the central cornea. It is believed that this technique might provide a simple, non-surgical method for treating myopia by inducing swelling in defined ring on the corneal periphery, thereby causing flattening of the central cornea.

Rejuvenation of Human Skin

### Example 4

### Acylation of Human Skin

Processed human skin was obtained for treatment. The processed skin was comprised of lyophilized dermis. Four samples were tested, two 2 cm×4 cm specimens and two 2 cm×8 cm specimens. Two sets of specimens were treated with 50 mg/mL of glutaric anhydride (dose #1) and the other two sets with 100 mg/mL of glutaric anhydride (dose #2). The rejuvenation agents were prepared in dilute buffer solution. Prior to treatment with the active agent, skin specimens were pre-treated with buffer alone. Treatments were completed in 1 minute. Results of the mechanical properties of the specimens are shown in the following Table 3.

TABLE 3
Effects Of Acylation Treatment On The Mechanical Properties Of
Dermis

| Specimen | Dose | Thickness | Stress (MPa) | Strain (%) |
|---|---|---|---|---|
| Untreated | -0- | 1.82 mm | 5.7 | 72 |
| Treated | 50 mg/mL | 1.77 mm | 2.5 | 79 |
| Untreated | -0- | 1.55 mm | 3.1 | 74 |
| Treated | 100 mg/mL | 2.55 mm | 2.7 | 84 |

As shown in Table 3, treatment with 100 mg/mL increased dermal thickness, decreased stress and increased strain. It is presumed that dermal thickness and strain (related to elasticity) increased due to increased water binding (hydration). The active agent was selected to produce an increase in dermis hydrophilicity.

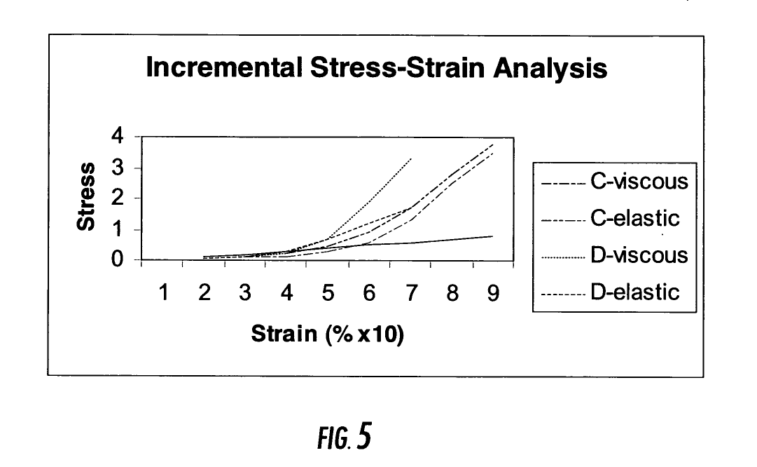

In a second experiment one strip (Sample C) of dermal tissue was exposed to a 100 mg/mL anhydride solution. A second strip (Sample D) was exposed to the same anhydride solution after about 2 minutes. At this point the active anhydride was significantly reduced as it hydrolyzed to acid form. The results are shown in FIG. 5.

As noted, both the elastic and viscous curves for Sample C were shifted to higher percent strain. This shift means that this Sample was better able to resist stress before failure. In practical terms, the anhydride treatment improved resistance to stress, characteristic of younger skin. In addition, Sample C was significantly thicker than Sample D, again a characteristic of younger skin. Overall, the acylation treatment appears to produce effects to rejuvenate older, thinner, and more brittle skin.

In a third series of experiments, reconstituted, lyophilized human dermis samples were exposed to the acylation agents (glutaric anhydride). Again, dermal thickness increased from about 1 mm to 2 mm due to increased water binding. In addition, dermal pliability was increased as evidenced by the decreased relaxation time. It is hypothesized that the reactive agent changes the net charge distribution on collagen fibrils reducing the intensity of intrafibrillar interactions and increasing pliability. Increased water binding and pliability are characteristics associated with young skin.

Treatment of Articular Cartilage

### Example 4

### Cartilage Rejuvenation—Chemical Treatments

Articular cartilage was dissected from both femoral and tibial chondyles of sacrificed rabbits. Cartilage slices were stored in sterile physiological saline. Eight slices of similar size (about 2 mm×2 mm) were placed in individual Eppendorf tubes. Physiological saline was replaced with 0.02M disodium phosphate at pH 8.5. Two specimens were used as Controls and did not receive chemical treatment. Three specimens were treated with 0.02M disodium phosphate containing 5 mg/ml glutaric anhydride (95%). The remaining three specimens were treated with 0.02M disodium phosphate containing 5 mg/ml of acetic anhydride. Samples were exposed to control solution or acylation chemical for 3 minutes and then washed three times with sterile phosphate buffered saline (0.04M) at pH 7.2. Results are reported in the following Table 4.

TABLE 4
Stress-Strain Analysis

| | Low | |
|---|---|---|
| Specimen | Region Modulus (Pa) | High Modulus Region Pa) |
| Control | 33.5 | 50.5 |
| Glutaric | 26.7 | 58.1 |
| anhydride | | |
| Acetic anhydride | 16.6 | 47.7 |

Results shown in Table 4 indicate that both glutaric and acetic anhydride reduced low modulus. The low modulus represents resistance to squeezing water from the matrix network. High modulus represents resistance to compression of the matrix itself. As shown, glutaric anhydride appeared to show an increase in high modulus indicating strengthening of the matrix network; acetic anhydride slightly reduced the high modulus indicating weakening of the matrix. While only preliminary, these in vitro results indicate that specific acylation can be performed to change the biophysical characteristics of articular cartilage. Histological examination showed no differences between control and treated articular cartilage.

Treatment of Presbyopia

### Example 5

### Softening Sclera Tissues

Bovine eyes were procured from a local slaughterhouse and stored in Optisol solution. Whole eyes were removed from the preservation solution and the sclera dried using Kim wipes. A solution of 0.2M disodium phosphate, pH 9.5 was applied to the dried sclera tissue. The tissue was exposed to the disodium phosphate solution for 1 minute. The tissue was again dried using a Kim wipe and a solution containing 100 mg/mL of glutaric anhydride in disodium tissue was exposed to the anhydride solution for 1 minute and the tissue then thoroughly rinsed with sterile 0.02M phosphate buffer, pH 7.2. The exposed tissue was examined for tissue softening. Compression analysis demonstrated that the exposed tissue had significant loss of low modulus. Sclera tissue softening was also obvious by application of thumb pressure to treated and untreated areas of the sclera tissue. It is proposed that sclera softening using acylating agents may be used to expand sclera tissue, thereby permitting more space for the ciliary body to expand and contract the lens. Sclera expansion has been shown to be effective in reversing presbyopia.

### OTHER EMBODIMENTS

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention herein. Such equivalents are intended to be encompassed in the scope of the present invention.

All references, including patents, publications, and patent applications, mentioned in this specification are herein incorporated by reference in the same extent as if each independent publication, patent or patent application was specifically and individually indicated to be incorporated by reference.

1. A method for treating animal tissue, comprising the steps of:
(a) providing an exposed surface area of animal tissue;
(b) applying an alkaline solution to the exposed tissue surface area to form a tissue protein having a net charge; and
(c) applying an acylating agent to the exposed tissue surface area so that the acylating agent reacts with the tissue protein to form a protein complex having a different net charge than the net charge of the tissue protein formed in above step (b).

2. The method of claim 1, further comprising the step of rinsing the exposed tissue surface area with a neutral or alkaline solution after step (c).

3. The method of claim 1, wherein the tissue protein is collagen.

4. The method of claim 1, wherein the animal tissue is human tissue.

5. The method of claim 1, wherein the animal tissue is human tissue and the human tissue is treated in vivo.

6. The method of claim 5, wherein the tissue is skin tissue.

7. The method of claim 6, wherein treatment with the acylating agent makes the skin tissue more hydrophilic.

8. The method of claim 5, wherein the tissue is cornea tissue.

9. The method of claim 8, wherein the acylating agent is applied to a peripheral area of a human cornea, thereby resulting in swelling of the peripheral area and flattening of a central area of the cornea.

10. The method of claim 5, wherein the tissue is sclera tissue.

11. The method of claim 3, wherein the tissue is conjunctival tissue.

12. The method of claim 5, wherein the tissue is articular cartilage.

13. The method of claim 1, wherein the alkaline solution comprises a compound selected from the group consisting of disodium phosphate, sodium pyrophosphate, and sodium borate.

14. The method of claim 11, wherein the alkaline solution has a pH in the range-of about 7.5 to about 9.5.

15. The method of claim 1, wherein the acylating agent comprises a compound selected from the group consisting of sulfonic acids, sulfonyl chlorides, and acid chlorides.

16. The method of claim 1, wherein the net charge of the tissue protein formed in step (b) is positive, and the net charge of the protein complex formed in step (c) is negative.

17. The method of claim 1, wherein the net charge of the tissue protein formed in step (b) is positive, and the net charge of the protein complex formed in step (c) is neutral.

18. The method of claim 1, wherein the tissue protein is treated with an acylating agent that causes the protein complex formed in step (c) to have increased net positive charges over the tissue protein formed in step (b).

19. The method of claim 1, wherein the tissue protein is treated with an acylating agent that causes the protein complex formed in step (c) to have increased net negative charges over the tissue protein formed in step (b).
